(19) United States
(12) Patent Application Publication
(10) Pub. No.: US 2017/0022058 A1
(43) Pub. Date: Jan. 26, 2017

(54) PROCESS AND SYSTEM FOR THE GENERATION OF SYNTHESIS GAS

(71) Applicants: Harald Ranke, Pocking (DE); Andreas Seliger, Munich (DE); Volker Göke, Geretsried (DE); Christine Kandziora, Baierbrunn (DE)

(72) Inventors: Harald Ranke, Pocking (DE); Andreas Seliger, Munich (DE); Volker Göke, Geretsried (DE); Christine Kandziora, Baierbrunn (DE)

(21) Appl. No.: 15/083,601

(22) Filed: Mar. 29, 2016

(30) Foreign Application Priority Data
Mar. 31, 2015 (DE) 102015004213.0

(51) Int. Cl. C01B 3/48; C07C 273/04; B01J 7/02; C10G 2/00; C01B 3/38; C01C 1/04

(52) U.S. Cl. CPC C01B 3/48; C01B 3/384; C01C 1/0441; C01C 1/0488; B01J 7/02; C10G 2/30; C07C 273/04; C01B 2203/0233; C01B 2203/0283; C01B 2203/062; C01B 2203/068; C01B 2203/0827; C01B 2203/1241

(57) ABSTRACT

A process and system for the generation of synthesis gas that is provided, in particular, for preparing hydrocarbon-containing fuel, ammonia or urea. The process follows the steps of providing a first feed gas stream of methane and reacting the first feed gas stream with steam in a reforming step, obtaining a synthesis gas stream of CO and H₂. It is further provided that at least one first substream is separated off from the feed gas stream before the reforming step, the first substream is then burnt with a second feed gas stream of at least 95% by volume oxygen to give an exhaust gas stream comprising CO₂ and water, and at least one part of the exhaust gas stream is recirculated to the feed gas stream after the first substream is separated off.

### CROSS REFERENCE TO RELATED APPLICATIONS

This application claims priority from German Patent Application DE 10 2015 004 213.0 filed on Mar. 31, 2015.

### BACKGROUND OF THE INVENTION

The invention relates to a process and a system for the generation of synthesis gas.

Such a process comprises the steps: providing a first feed gas stream comprising methane and reacting the first feed gas stream with steam in a reforming step, obtaining a synthesis gas stream comprising CO and H₂.

The steam methane reforming (SMR) technology is a known and established technology that has been used for decades to prepare synthesis gas. The SMR technology is primarily particularly suitable, in comparison with other processes, for hydrogen production, wherein the hydrogen content, which is in any case already high, of the synthesis gas obtainable by SMR can be additionally increased by a watergas-shift reaction.

For the preparation of synthesis gas per se, however, other techniques, such as, for instance, partial oxidation (PDX) or autothermal reforming (ATR), have also proved to be at least competitive, if not even more suitable than the SMR technology, in particular in the preparation of large amounts, in which unwanted by-products of the SMR technology can prove to be disadvantageous. On the other hand, PDX or ATR processes require oxygen, wherein the oxygen demand increases with the hydrogen content of the synthesis gas.

On the basis of this background, it is the object of the invention to provide a simple and economic process for the preparation of synthesis gas that is characterized in particular by a high carbon efficiency and reduced carbon dioxide emission,

### SUMMARY OF THE INVENTION

This object is achieved in that a first substream is separated off from the feed gas stream before the reforming step, which first substream is then reacted with a second feed gas stream comprising at least 95% by volume oxygen to give an exhaust gas stream comprising CO₂ and water, and at least one part of the exhaust gas stream is recirculated to the feed gas stream after the first substream is separated off.

Advantageously, the reforming step is carried out in a steam reformer, wherein the steam reformer comprises at least one reformer tube and a combustion chamber and is designed to transfer the heat arising in the combustion chamber to the at least one reformer tube. The reformer tube is configured with a suitable catalyst, such as, for example, a nickel catalyst.

Recycling the at least one part of the exhaust gas results in a plurality of advantages: the yield of synthesis gas is increased, the CO₂ emission is reduced, and the carbon efficiency of the process is increased. In addition, no import of external CO₂ is necessary in order to shift the H₂/CO ratio if desired.

The exhaust as stream is, in particular, a CO₂-rich gas stream that has a CO₂ content of at least 50% by volume, in partcular a CO₂ content from 54% by volume to 75% by volume, preferably a CO₂ content of 61% by volume. Such a gas stream can advantageously be used in processes requiring CO₂, such as, for example, for synthesizing urea or for enhanced oil recovery, wherein CO₂ is conducted at high pressure into a borehole in order to force the oil to the surface.

According to an embodiment of the invention, it is provided that the feed gas stream is desulphurized before the reforming step and before the first substream is separated off, wherein sulphur-containing compounds are removed from the feed gas stream.

According to an embodiment of the invention, it is provided that the reforming step is carried out at a temperature from 750° C. to 950° C. and at a pressure from 10 bar to 40 bar, preferably at a pressure of 30 bar.

According to an embodiment of the invention, it is provided that the heat arising in the combustion of the first substream is transferred to the reforming step. Advantageously, heat is thereby provided for the endothermic reforming step.

According to a further embodiment of the invention, it is provided that another part of the exhaust gas stream is recirculated to the first substream. Advantageously, firstly the flame temperature can be controlled thereby. Secondly, the enrichment of the combustion chamber with CO₂ leads to the heat being transferred more efficiently to the reforming step or to the reformer tube as a result of the now increased IR radiation of the CO₂ molecules.

According to a further embodiment of the invention, it is provided that the second feed gas stream comprising at least 95% by volume oxygen is provided by the gas separation of air. In particular, a third feed gas stream that substantially comprises nitrogen is provided by the abovementioned gas separation. A feed gas stream substantially comprising nitrogen is, in the context of the present description, a gas stream which has a nitrogen content of at least 98% by volume, 99% by volume, 99.9% by volume, or 99.99% by volume.

According to a further embodiment of the invention, it is provided that the synthesis gas stream comprising CO and H₂ is cooled to a temperature from 50° C. to 70° C., with generation of steam. Advantageously, the steam required for the reforming step is provided by this embodiment. The steam generated, however, can advantageously also be used in other processes, such as, for instance, for power generation. According to a further embodiment of the invention, it is provided that the synthesis gas stream comprising CO and H₂ is then further heated to a temperature from 150° C. to 220° C.

According to a further embodiment of the invention, it is provided that at least one second substream is separated off from the synthesis gas stream comprising CO and H₂, which at least one second substream is reacted in a watergas-shift reaction step to give a crude hydrogen stream, wherein CO and water are reacted to give CO₂ and hydrogen. Advantageously, a larger amount of elemental hydrogen is provided thereby that can be used for a multiplicity of processes, such as, for instance, ammonia synthesis.

According to a further embodiment of the invention, it is provided that a first tail gas stream which substantially comprises CO₂, and, optionally, H₂, unreacted CO and/or unreacted methane, is separated off from the crude hydrogen stream, with a first product stream which substantially comprises H₂ being obtained, and the first tail gas stream is recirculated to the first substream. A product stream substantially comprising H₂ is, in the context of the invention, characterized by a hydrogen content of at least 99% by volume, 99.9% by volume, or 99.99% by volume.

According to a further embodiment of the invention, it is provided that the first tail gas stream is separated off by pressure-swing adsorption, using a substantially H₂-containing purge gas, wherein the tail gas stream additionally comprises H₂. Advantageously, at least one part of the H₂-containing purge gas is provided by the first product gas. A substantially H₂-comprising purge gas, in the context of the invention, denotes a gas stream that has a hydrogen content of at least 98% by volume, 99% by volume, 99.9% by volume, or 99.99% by volume. Advantageously, the hydrogen present in the tail gas stream can be recirculated to the combustion chamber of the reformer, in order there to generate by combustion the heat that is required for the reforming step.

According to a further embodiment of the invention, it is provided that the synthesis gas stream comprising CO and H₂ is reacted in a Fischer-Tropsch synthesis step to give a crude product stream comprising a mixture of at least one light C₁-C₄ hydrocarbon, at least one heavy hydrocarbon having more than 4 carbon atoms, and unreacted synthesis gas comprising CO and H₂.

According to a further embodiment of the invention, it is provided that a second tail gas stream comprising the at least one light C₁-C₄ hydrocarbon and the unreacted synthesis gas is separated off from the crude product stream, and the second tail gas stream is recirculated to the feed gas stream. Advantageously, the second tail gas stream is conducted without further workup directly into the feed gas stream. As a result of this dilution of the feed gas stream by the second tail gas stream, the CO partial pressure in the feed gas is reduced, which markedly increases the service life of the catalyst in the reformer tube.

According to a further embodiment of the invention, it is provided that a second substream is separated off from the second tail gas stream, and the second substream is recirculated to the Fischer-Tropsch synthesis step. Advantageously, the yield of the Fischer-Tropsch synthesis step can be increased thereby, since the unreacted synthesis gas is fed back to the reaction.

According to a further embodiment of the invention, it is provided that a third substream is separated off from the second tail gas stream, which third substream is burnt generating steam, wherein the resultant exhaust gas of the combustion is at least in part recirculated to the reforming step, or to the steam reformer, in particular via the second tail gas stream.

According to a further embodiment of the invention, it is provided that the first product stream comprising substantially H₂ is conducted at least in part into the crude product stream, in particular into the crude product stream after the second tail gas stream is separated off. Advantageously, oxygen-containing or unsaturated hydrocarbons can be reduced to saturated hydrocarbons thereby.

According to an alternative embodiment of the invention, it is provided that the first product stream substantially comprising H₂ is provided for the synthesis of ammonia, wherein the synthesized ammonia is provided, in particular, for synthesizing urea.

According to a further alternative embodiment of the invention, it is provided that the third feed gas stream comprising substantially nitrogen is provided for synthesizing ammonia.

According to a further alternative embodiment, provision is made for reacting ammonia with CO₂ to give urea. Advantageously, CO₂ here is provided from a part of the exhaust gas stream.

According to a further aspect of the invention, a process for the preparation of ammonia is provided. Such a process comprises the steps:

providing a first feed gas stream comprising methane,

reacting the first feed gas stream with steam in a reforming step, with a synthesis gas stream comprising CO and H₂ being obtained, wherein
at least one first substream is separated off from the first feed gas stream before the reforming step,
the first substream is then burnt with a second feed gas stream comprising at least 95% by volume oxygen to give an exhaust gas stream comprising CO₂ and water,
at least one part of the exhaust gas stream is recirculated to the first feed gas stream upstream of the reforming step,

reacting at least one part of the synthesis gas stream comprising CO and H₂ in a watergas-shift reaction to give a crude hydrogen stream, wherein CO and water are reacted to give CO₂ and H₂, and optionally separating off a first product stream comprising substantially H₂ from the crude hydrogen stream,

reacting hydrogen and nitrogen to give ammonia, wherein the hydrogen is provided by the crude hydrogen stream or first product stream comprising substantially H₂.

According to a further embodiment of the invention, it is provided that the second feed gas stream is provided by the gas separation of air, wherein a third feed gas stream substantially comprising nitrogen is additionally provided by the gas separation, and the above-described nitrogen provided for the ammonia production is provided by the third feed gas stream.

According to a further embodiment of the invention, it is provided that, from the crude hydrogen stream, in addition to the first product stream, a first tail gas stream is separated off that substantially comprises CO₂ and, optionally, H₂, unreacted CO and/or unreacted methane, and the first tail gas stream is recirculated to the first substream.

According to a further embodiment of the invention, it is provided that the first tail gas stream is separated off by pressure-swing adsorption, using an H₂-containing purge gas, in such a manner that the tail gas stream additionally comprises H₂.

Advantageously, the hydrogen present in the tail gas stream can be recirculated to the combustion chamber of the reformer, in order there to generate, by combustion, the heat required for the reforming step.

According to a further aspect of the invention, a process for the preparation of urea is provided. The process comprises the steps:

providing a first feed gas stream comprising methane,

reacting the first feed gas stream with steam in a reforming step, with a synthesis gas stream comprising CO and H₂ being obtained, wherein
at least one first substream is separated off from the first feed gas stream before the reforming step,
the first substream is then burnt with a second feed gas stream comprising at least 95% by volume oxygen to give an exhaust gas stream comprising CO₂ and water,
at least one part of the exhaust gas stream is recirculated to the first feed gas stream upstream of the reforming step,
reacting at least one part of the synthesis gas stream comprising CO and H₂ in a watergas-shift reaction to give a crude hydrogen stream, wherein CO and water are reacted to give CO₂ and H₂, and optionally separating off a first product stream comprising substantially H₂ from the crude hydrogen stream,
reacting hydrogen and nitrogen to give ammonia, wherein the hydrogen is provided by the crude hydrogen stream or first product stream comprising substantially H₂,
reacting the ammonia and CO₂ to give urea.

In this case, urea is formed in accordance with the following reactions:

NH₃+CO₂⇄H₂NCOONH₄

H₂NCOONH₄⇄(NH₂)₂CO+H₂O

According to a further embodiment of the invention, it is provided that the second feed gas stream is provided by the gas separation of air, wherein a third feed gas stream substantially comprising nitrogen is additionally provided by the gas separation, and the above-described nitrogen provided for the ammonia preparation is provided by the third feed gas stream.

According to an embodiment of the invention, the CO₂ is provided by the exhaust gas stream. According to a further embodiment of the invention, unreacted oxygen is removed from the exhaust gas stream. According to an embodiment of the invention, the oxygen is separated off catalytically, or is removed from the exhaust gas stream by reaction with a reducing agent. According to an embodiment of the invention, the hydrogen provided by the crude hydrogen stream or first product stream is used as reducing agent.

According to a further embodiment of the invention, it is provided that, in addition to the first product stream, a first tail gas stream is separated off from the crude hydrogen stream, which first tail gas stream substantially comprises CO₂ and, optionally, H₂, unreacted CO and/or unreacted methane, and the first tail gas stream is recirculated to the first substream. According to a further embodiment of the invention, it is provided that the first tail gas stream is separated off by pressure-swing adsorption, using an H₂-containing purge gas, in such a manner that the tail gas stream additionally comprises H₂. Advantageously, the hydrogen present in the tail gas stream can be recirculated to the combustion chamber of the reformer, in order there to generate, by combustion, the heat that is required for the reforming step.

According to a further aspect of the invention, a system for synthesis gas preparation is provided. Such a system comprises:

a piping system which is designed for conducting a feed gas stream,

a steam reformer that is flow-connected to the piping system, which steam reformer comprises at least one reformer tube and a combustion chamber, wherein the combustion chamber is designed to burn a gas stream comprising a fuel in the presence of an oxygen-containing gas stream with an exhaust gas stream being formed and to transfer the resultant heat to the at least one reformer tube,

wherein the piping system is additionally designed to separate the feed gas stream into a feed gas main stream and a feed gas substream, to conduct the feed gas main stream into the at least one reformer tube, to conduct the feed gas substream into the combustion chamber, and to conduct the exhaust gas stream into the feed gas main stream. The at least one reformer tube is equipped with a catalyst, for example with a catalyst comprising nickel or ruthenium.

According to an embodiment of the invention, it is provided that the steam reformer is flow-connected to a Fischer-Tropsch reactor, which is designed to convert synthesis gas to hydrocarbons. In particular, such a reactor comprises at least one catalyst that is based, in particular, on a transition metal such as, for instance, cobalt, iron, nickel or ruthenium.

According to a further embodiment of the invention, it is provided that the Fischer-Tropsch reactor is flow-connected to a separation unit that is designed to separate off a tail gas stream comprising light C₁-C₄ hydrocarbons and synthesis gas from a hydrocarbon-containing material stream. Advantageously, the piping system is designed to conduct the tail gas stream into the Fischer-Tropsch reactor and/or into the feed gas main stream.

According to a further embodiment of the invention, it is provided that the steam reformer is flow-connected to a watergas-shift reactor which is designed to convert CO and water to H₂ and CO₂.

According to a further embodiment of the invention, the watergas-shift reactor is flow-connected to a pressure-swing adsorber which is designed to separate off a further tail gas stream comprising CO₂ and optionally H₂, CO and/or methane from a hydrogen-containing gas stream. Advantageously, the piping system is designed to conduct the further tail gas stream into the feed gas substream or into the combustion chamber.

According to a further embodiment of the invention, it is provided that the separation unit is flow-connected to a hydrogenation reactor which is designed to hydrogenate unsaturated or oxygen-containing hydrocarbon compounds.

According to a further embodiment, the hydrogenation reactor is flow-connected to the pressure-swing adsorber.

According to a further embodiment of the invention, between the steam reformer and the Fischer-Tropsch reactor and/or between the steam reformer and the watergas-shift reactor, a heat exchanger is arranged which is designed to transfer heat from a hot gas stream to water, with the formation of steam. In particular, the heat exchanger comprises at least one tube that is enclosed by a shell, wherein the water that is to be heated is conducted preferably on the tube side and the hot gas stream on the shell side.

According to a further embodiment of the invention, the combustion chamber is flow-connected to an air separation unit that is designed to separate air into oxygen and nitrogen.

According to an alternative embodiment of the invention, the watergas-shift reactor or the pressure-swing adsorber is flow-connected to an ammonia reactor, wherein the ammonia reactor is designed to react hydrogen and nitrogen to form ammonia.

According to a further embodiment, the ammonia reactor is flow-connected to the air separation unit.

According to a further embodiment of the invention, the ammonia reactor is flow-connected to a urea reactor that is designed to react ammonia with carbon dioxide to give urea.

According to a further embodiment of the invention, the combustion chamber is flow-connected to the urea reactor.

According to an embodiment of the invention, an oxygen separator is arranged between the combustion chamber and the urea reactor, which oxygen separator is designed to remove oxygen from the exhaust gas of the combustion chamber. In particular, the oxygen separator is designed to permit oxygen to combust with hydrogen to form water.

### BRIEF DESCRIPTION OF THE DRAWINGS

Further details and advantages of the invention will be explained by the following descriptions of figures of exemplary embodiments with reference to the figures.

In the drawings:

FIG. 1 shows a diagram of an embodiment of the process according to the invention;

FIG. 2 shows a diagram of an alternative embodiment of the process according to the invention; and

FIG. 3 shows a diagram of a further alternative embodiment of the process according to the invention.

### DETAILED DESCRIPTION OF THE INVENTION

### EXAMPLES

### Example 1

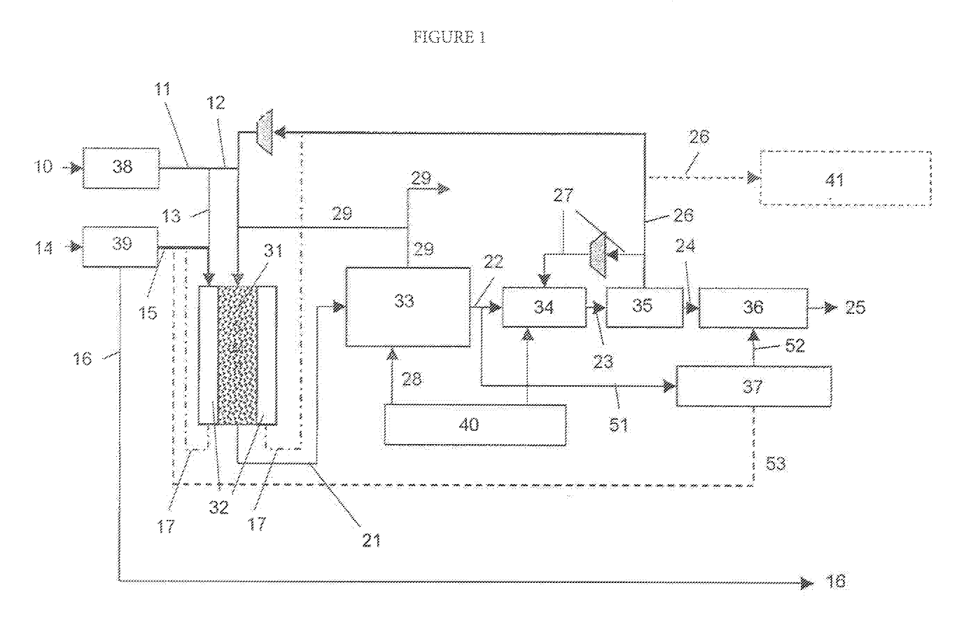

FIG. 1 illustrates the main steps of a preferred procedure of the invention, a Fischer-Tropsch (FT) process, in which gaseous hydrocarbons are converted to liquid hydrocarbons. The steps comprise substantially a desulphurization 38 of the feed gas stream 10, a reforming 31 of the feed gas stream 11, 12, a cooling of the reformate 21, and a Fischer-Tropsch synthesis 34 with the cooled reformate 22.

After the desulphurization 38 in which sulphur-containing compounds are removed from a hydrocarbon-containing feed gas stream 10 such as, for example, natural gas, the desulphurized feed gas stream 11 is separated into a feed gas main stream 12 (also designated first feed gas stream) and a feed gas substream 13 (also designated substream). The feed gas main stream 12 is conducted into a steam reformer 31 which comprises a combustion chamber 32 and a plurality of reformer tubes 31. The reformer tubes 31 in this case are equipped with one or more suitable catalysts such as, for instance, a nickel catalyst or a ruthenium catalyst. The feed gas main stream 12 flows together with steam 29 into the reformer tubes 31 and is there reacted to give a synthesis gas 21 comprising CO and H₂, in particular at a temperature from 750° C. to 950° C., and a pressure from about 10 bar to 40 bar, preferably at a pressure of 30 bar.

The synthesis gas 21 from the reformer tubes 31 is cooled 33, in particular firstly to a temperature from 50° C. to 70° C., wherein water 28 from a reservoir 40 is heated by the heat withdrawn with formation of saturated steam 29. The cooled synthesis gas 22 is then dried in a separator and then heated back to a temperature from 150° C. to 220° C. The dry synthesis gas 22 is then passed into a Fischer-Tropsch reactor 34 in which CO and hydrogen are reacted to give a crude product 23 comprising hydrocarbons of differing chain lengths. Typically, the crude product 23 contains light hydrocarbons or liquid gases, crude benzene, diesel and paraffins, and also unreacted synthesis gas 22. The unreacted synthesis gas and the light hydrocarbons are separated off from the crude product 23 of the Fischer-Tropsch synthesis 34 and recycled via two different tail gas streams 26, 27 (F-T tail gas): an internal tail gas stream 27 to the Fischer-Tropsch reactor, and an external tail gas stream 26 back into the steam reformer 31.

Preferably, a part of the tail gas stream 26 is separated off. As a result, advantageously, accumulations of inert compound in the above-described recycling circuit can be avoided. The part separated off in this case is advantageously used as fuel (41) or profitably burnt.

The feed gas substream 13 is conducted into the combustion chamber 32 of the steam reformer and burnt with oxygen 15 as oxidizing agent. The resultant flue gas or exhaust gas 17 of the combustion, which principally contains CO₂ and water and traces of H₂, is recirculated in one part into the reformer 31 or the feed gas main stream 12, in particular via the external tail gas stream 26 from the Fischer-Tropsch synthesis 34. The other part of the exhaust gas 17 is recirculated into the combustion chamber 32 or the feed gas substream 13, in order to regulate the flame temperature.

A part 51 of the cooled synthesis gas is conducted into a watergas-shift reactor 37, in which CO is reacted to give CO₂ and hydrogen, with addition of steam. The hydrogen 52 is then separated off via a pressure-swing adsorption (PSA) 37 as hydrogen-containing product stream 52, and used for hydrogenation 36 of the products 24 of the Fischer-Tropsch synthesis 34. The tail gas 53 resulting from the pressure-swing adsorption is used as fuel in the combustion chamber 32 of the steam reformer. Advantageously, the pressure-swing adsorption 37 is carried out using a hydrogen-containing purge gas that advantageously is provided at least in part by the hydrogen-containing product stream 52. By using the hydrogen-containing purge gas, the tail gas 53, in addition to CO₂, also contains considerable amounts of hydrogen which, as described above, can be used as fuel for the steam reformer.

The proposed procedure increases the carbon efficiency of the process. In addition, the proposed procedure permits a more effective utilization of the resultant heat.

Further advantages are:
recirculating the tail gas from the Fischer-Tropsch synthesis (FT tail gas) without further workup and/or the exhaust gas to the synthesis gas step reduces the partial pressure of CO which is responsible for the beta-deactivation of the nickel catalyst;
recirculating the exhaust gas increases the yield of the synthesis gas step, that is to say more H₂ and CO molecules are formed per unit volume of the natural gas used;
no energy export;
lower CO₂ emission.

The table below summarizes some key parameters which were achieved by a conventional SMR process (standard) and by the proposed procedure (oxy-comb). The comparison was made on the basis of the same natural gas.

| | Process | |
|---|---|---|
| | Standard | Oxy-Comb |
| Natural gas feed (Ncum/h) | 43 500 | 43 500 |
| Fuel feed (Ncum/h) | 4000 | 800 |
| Total (Ncum/h) | 47 500 | 44 300 |
| Oxygen (Ncum/h) | 0 | 42 000 |
| Tail gas fuel | 29 000 | 16 900 |
| SMR fired duty (MW) | 117 | 134.3 |
| Product | | |
| H₂ + CO (Ncum/h) | 71 383 | 121 913 |
| Net energy (MW) | 15.1 | −11.2 |
| Natural gas equivalents | 1991.0 | −1477.0 |
| Nitrogen product (Ncum/h) | 0.00 | 4848 |
| CO₂ Emission (MTPD) | 45 510 | 45 776 |
| | 967.0 | 663.0 |
| Ratio of synthesis gas generated/natural | 2.0 | 2.7 |
| gas used | | |

### Example 2

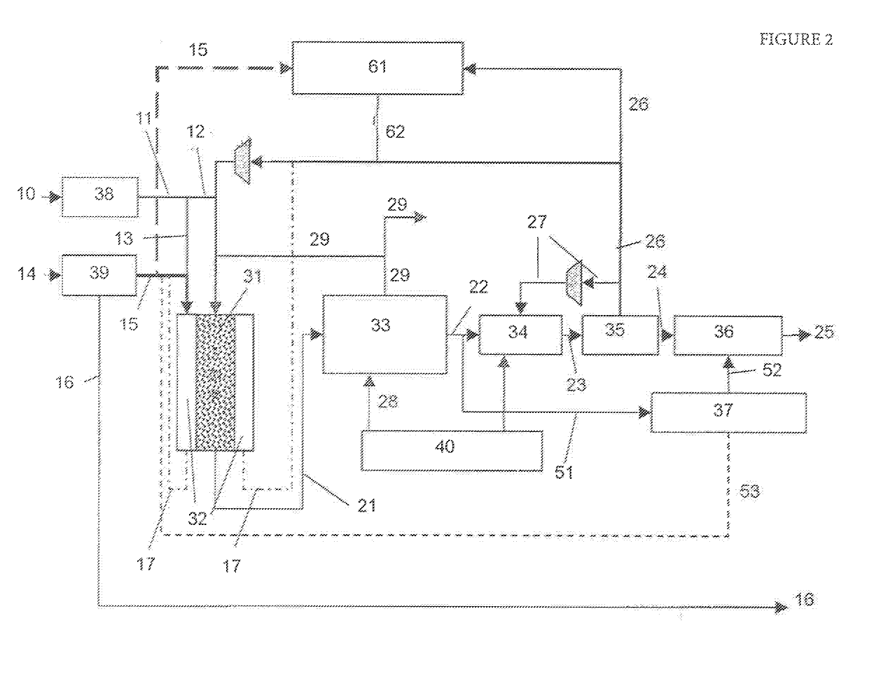

FIG. 2 illustrates a further development of the procedure according to the invention from Example 1. It is accordingly provided that a part of the tail gas 26 which is separated off from the crude product 23 of the Fischer-Tropsch synthesis 34 is burnt 61 for energy generation, wherein steam 29 is provided for the energy generation by the combustion 61. The flue gases and exhaust gases 62 formed in this combustion 61 are, after water separation, recirculated to the reformer 31 or the feed gas main stream 12 or the tail gas stream 26 from the Fischer-Tropsch synthesis 34. Alternatively, a part of the feed gas substream 13 can also be used for this purpose. Advantageously, the process can be operated in an energy-neutral manner by such a process.

### Example 3

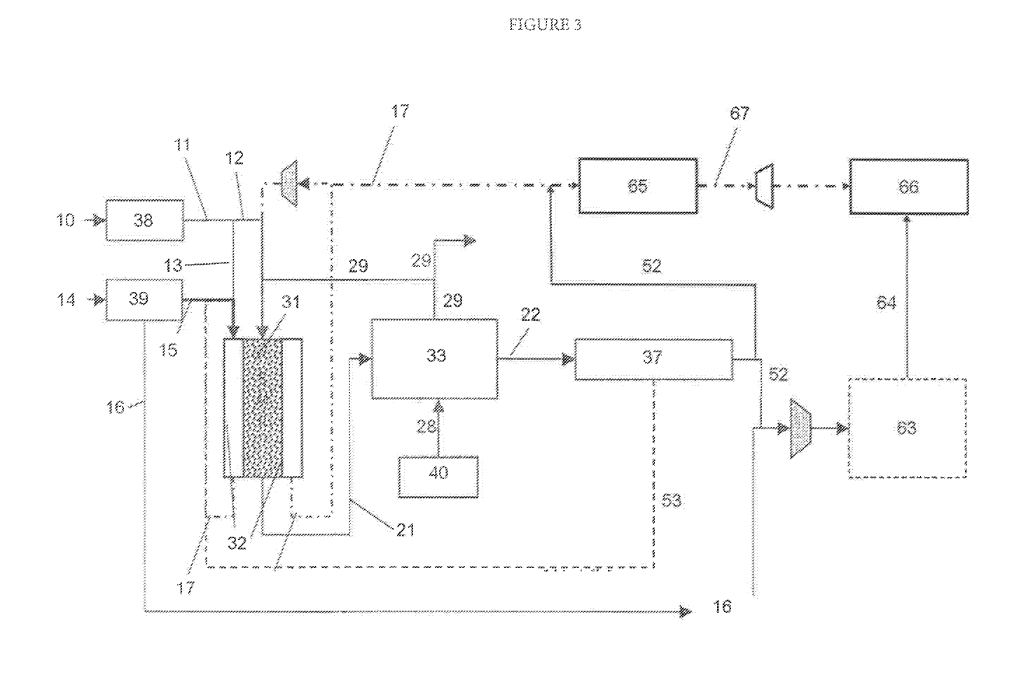

The process according to the invention can advantageously also be used in processes other than the Fischer-Tropsch synthesis 34. FIG. 3 shows an alternative embodiment of the invention, wherein the synthesis gas 22 that is generated is exclusively used in a watergas-shift reaction 37. The resultant hydrogen-rich product gas 52 in this case is then used in the synthesis 63 of ammonia 64 in which elemental hydrogen 52 and nitrogen 16 are reacted to give ammonia 64. Advantageously, the elemental nitrogen 16 necessary therefor is provided by the above-described air separation 39.

The synthesized ammonia 64 can further be reacted to give urea, wherein the carbon dioxide required therefor is advantageously provided by one of the above-described exhaust gas streams 17. The exhaust gas stream 17, in addition to CO₂, also still contains oxygen which is unreacted in the combustion 32 at a concentration of typically 1% by volume to 3% by volume. The oxygen, before use of the exhaust stream 17, is separated off 65 in the urea synthesis 66 in order to avoid disadvantageous reactions of the oxygen during urea synthesis. In this case the oxygen is either separated off catalytically, or is reacted with a reducing agent such as hydrogen 52, wherein the water formed in the latter case is then separated off. The then substantially oxygen-free, CO₂-containing, gas stream 67 is then compressed and reacted 66 with the synthesized ammonia 64 to give urea.

### LIST OF REFERENCE SIGNS

| | |
|---|---|
| 10 | Natural gas |
| 11 | Desulphurized natural gas or first feed gas stream |
| 12 | Feed gas main stream or first feed gas stream |
| 13 | Feed gas substream or substream |
| 14 | Air |
| 15 | Gas stream containing 95% by volume to 99% by volume oxygen |
| 16 | Nitrogen |
| 17 | Flue gas/exhaust gas |
| 21 | Hot crude synthesis gas |
| 22 | Cooled crude synthesis gas |
| 23 | F-T (Fischer-Tropsch) crude product |
| 24 | F-T crude product without synthesis gas and light hydrocarbons |
| 25 | Hydrogenated F-T product |
| 26 | Tail gas/F-T tail gas (synthesis gas and light hydrocarbons) |
| 27 | Recycling stream in F-T synthesis |
| 28 | Water |
| 29 | Steam |
| 31 | Reformer tubes |
| 32 | Combustion chamber |
| 33 | Heat exchanger |
| 34 | Fischer-Tropsch reactor |
| 35 | Separation unit |
| 36 | Hydrogenation reactor |
| 37 | Watergas-shift reactor/PSA |
| 38 | Desulphurization unit |
| 39 | Air separation unit |
| 40 | Water reservoir |
| 41 | Fuel system |
| 51 | Cooled synthesis gas stream |
| 52 | Hydrogen |
| 53 | Tail gas of the PSA |
| 61 | Auxiliary boiler |
| 62 | Exhaust gas from the auxiliary boiler |
| 63 | Ammonia synthesis |
| 64 | Ammonia |
| 65 | Oxygen removal |
| 66 | Urea synthesis |
| 67 | Substantially oxygen-free exhaust gas |

What we claim is:

1. A process for the generation of synthesis gas, comprising the steps:
providing a first feed gas stream comprising methane,
reacting the first feed gas stream with steam in a reforming step, obtaining a synthesis gas stream comprising CO and H₂,
characterized in that
at least one first substream is separated off from the first feed gas stream before the reforming step,
the first substream is then burnt with a second feed gas stream comprising at least 95% by volume oxygen to give an exhaust gas stream comprising CO₂ and water, and
at least one part of the exhaust gas stream is recirculated to the first feed gas stream upstream of the reforming step.

2. The process according to claim 1, characterized in that the heat arising in the combustion of the first substream is transferred to the reforming step.

3. The process according to claim 1, characterized in that another part of the exhaust gas stream is recirculated to the first substream.

4. The process according to claim 1, characterized in that the second feed gas stream is provided by the gas separation of air, wherein a third feed gas stream substantially comprising nitrogen is additionally provided by the gas separation.

5. The process according to claim 1, characterized in that the synthesis gas stream comprising CO and H₂ is cooled, with generation of steam.

6. The process according to claim 1, characterized in that at least one second substream is separated off from the synthesis gas stream comprising CO and H₂, which at least one second substream is reacted in a watergas-shift reaction step to give a crude hydrogen stream, wherein CO and water are reacted to give CO₂ and H₂.

7. The process according to claim 6, characterized in that a first tail gas stream which comprises CO₂, and H₂, unreacted CO and/or unreacted methane, is separated off from the crude hydrogen stream, with a first product stream which substantially comprises H₂ being obtained, and the first tail gas stream is recirculated to the first substream, wherein the first tail gas stream is separated off by pressure-swing adsorption, using an H₂-containing purge gas, in such a manner that the tail gas stream additionally comprises H₂.

8. The process according to claim 1, characterized in that the synthesis gas stream comprising CO and H₂ is reacted in a Fischer-Tropsch synthesis step to give a crude product stream that comprises a mixture of at least one light C₁-C₄ hydrocarbon, at least one heavy hydrocarbon having more than 4 carbon atoms, and unreacted synthesis gas comprising CO and H₂.

9. The process according to claim 8, characterized in that a second tail gas stream comprising the at least one light C₁-C₄ hydrocarbon and the unreacted synthesis gas is separated off from the crude product stream, and the second tail gas stream is recirculated to the first feed gas stream.

10. The process according to claim 9, characterized in that a second substream is separated off from the second tail gas stream, and the second substream is recirculated to the Fischer-Tropsch synthesis step.

11. The process according to claim 7, characterized in that the first product stream comprising substantially H₂ is conducted at least in part into the crude product stream after the second tail gas stream is separated off.

12. The process according to claim 9, characterized in that a third substream is separated off from the second tail gas stream, which third substream is burnt generating steam, wherein the resultant exhaust gas of the combustion is at least in part recirculated to the reforming step via the second tail gas stream.

13. A process for the preparation of ammonia, comprising the steps:
providing a first feed gas stream comprising methane,
reacting the first feed gas stream with steam in a reforming step, with a synthesis gas stream comprising CO and H₂ being obtained, wherein
at least one first substream is separated off from the first feed gas stream before the reforming step,
the first substream is then burnt with a second feed gas stream comprising at least 95% by volume oxygen to give an exhaust gas stream comprising CO₂ and water,
at least one part of the exhaust gas stream is recirculated to the first feed gas stream upstream of the reforming step, and
optionally, the second feed gas stream is provided by the gas separation of air, wherein a third feed gas stream substantially comprising nitrogen is additionally provided by the gas separation,
reacting at least one part of the synthesis gas stream comprising CO and H₂ in a watergas-shift reaction to give a crude hydrogen stream, wherein CO and water are reacted to give CO₂ and H₂, and optionally separating off a first product stream comprising substantially H₂ from the crude hydrogen stream,
reacting hydrogen and nitrogen to give ammonia, wherein the hydrogen is provided by the crude hydrogen stream or first product stream, and the nitrogen is provided by the third feed gas stream.

14. A process for the preparation of urea, comprising the steps:
providing a first feed gas stream comprising methane,
reacting the first feed gas stream with steam in a reforming step, with a synthesis gas stream comprising CO and H₂ being obtained, wherein
at least one first substream is separated off from the first feed gas stream before the reforming step,
the first substream is then burnt with a second feed gas stream comprising at least 95% by volume oxygen to give an exhaust gas stream comprising CO₂ and water,
at least one part of the exhaust gas stream is recirculated to the first feed gas stream upstream of the reforming step, and
optionally, the second feed gas stream is provided by the gas separation of air, wherein a third feed gas stream substantially comprising nitrogen is additionally provided by the gas separation,
reacting at least one part of the synthesis gas stream comprising CO and H₂ in a watergas-shift reaction to give a crude hydrogen stream, wherein CO and water are reacted to give CO₂ and H₂, and optionally separating off a first product stream comprising substantially H₂ from the crude hydrogen stream,
reacting hydrogen and nitrogen to give ammonia, wherein the hydrogen is provided by the crude hydrogen stream or first product stream, and the nitrogen is provided by the third feed gas stream, and
reacting the ammonia and CO₂ to give urea, wherein CO₂ is provided by the exhaust gas stream, wherein unreacted oxygen that is situated in the exhaust gas stream is reduced by hydrogen to form water, and wherein the hydrogen for the reduction of the oxygen is provided by the crude hydrogen stream or first product stream.

15. A system for synthesis gas preparation comprising
a piping system which is designed for conducting a feed gas stream,
a steam reformer that is flow-connected to the piping system, which steam reformer comprises at least one reformer tube and a combustion chamber, wherein the combustion chamber is designed to burn a gas stream comprising a fuel in the presence of an oxygen-containing gas stream with an exhaust gas stream being formed and to transfer the resultant heat to the at least one reformer tube,
characterized in that
the piping system is additionally designed to separate the feed gas stream into a feed gas main stream and a feed gas substream, to conduct the feed gas main stream into the at least one reformer tube, to conduct the feed gas substream into the combustion chamber, and to conduct the exhaust gas stream into the feed gas main stream.
